(19) United States
(12) Patent Application Publication
(10) Pub. No.: US 2025/0204992 A1
(43) Pub. Date: Jun. 26, 2025

(54) METHOD AND SYSTEM FOR 3D SURFACE MODEL FITTING VIA ROBOT ARM CONTROLLED LASER GRID AND CAMERA

(71) Applicant: EDDA TECHNOLOGY, INC., PRINCETON, NJ (US)

(72) Inventors: Yifan Wang, West Windsor, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 18/999,620

(22) Filed: Dec. 23, 2024

Related U.S. Application Data
(60) Provisional application No. 63/613,465, filed on Dec. 21, 2023.

(51) Int. Cl. A61B 34/20; B25J 9/16; G06T 7/521; G06T 7/73; G06T 19/20

(52) U.S. Cl. CPC A61B 34/20; B25J 9/1679; G06T 7/521; G06T 7/75; G06T 19/20; A61B 2034/2051; A61B 2034/2065; G06T 2207/10028; G06T 2207/30204; G06T 2210/41; G06T 2219/2004

(57) ABSTRACT

The present teaching relates to generating a point cloud of a target organ using a camera and a laser emitter inserted into a patient's body near the target organ and tracked by a tracker. The laser emitter emits, at tracked moving locations, laser beams that hit the target organ at multiple points which are captured by the camera in an image. A three-dimensional (3D) coordinate for each point is determined based on a laser line equation, connecting the laser emitter and the point, and an image line equation, connecting the camera and the point. The 3D coordinates for the multiple points are used to generate the point cloud.

### CROSS REFERENCE TO RELATED APPLICATION

The present application claims priority to U.S. Provisional Patent Application No. 63/613,465 filed Dec. 21, 2023, entitled “3D SURFACE MODEL FITTING BASED ON ROBOT-ARM CONTROLLED LASER GRID AND CAMERA”, which is incorporated herein by reference in its entirety.

### BACKGROUND

### 1. Technical Field

The present teaching generally relates to computers. More specifically, the present teaching relates to signal processing.

### 2. Technical Background

Knowing spatial information of a three-dimensional (3D) object is an essential need for different applications in various industries. For instance, the 3D location, the 3D shape, and the volumetric measurement of a target object may be determined first before controlling a robot to grab and move the target object. Different sensors and devices have been developed to obtain 3D spatial information of an object, including LiDAR sensors, depth cameras, as well as 3D scanners. Such traditional solutions usually all come with a high cost and have varying accuracy, reliability, and applicability in different scenarios. For instance, LiDAR sensors may be deployed in an unmanned vehicle as a sensor to detect the distances to surrounding obstacles and have gain popularity in recent years.

However, some applications may have requirements on such sensors, making the traditional solutions impossible or entirely inappropriate to be used to acquire 3D information about a target object. One typical scenario is the medical field. For example, during in a minimally invasive surgery, 3D information of an organ is needed in order to enable a robot arm to control a surgical instrument to approach a certain position on the organ to perform an operation. An example operation is resection of a target organ or a portion thereof. In this case, it is inappropriate to use traditional 3D sensor solutions because of their respective physical features. Although multiple laparoscopic cameras may be deployed for determining 3D information of an organ, it is difficult to achieve the goal because a laparoscopic camera has only a limited field of view and it can move around in a quite restricted space. Thus, it remains a significant challenge to acquire 3D information of a target organ during a medical procedure to obtain useful guidance to a surgical team. In addition, some target organs, such as a liver, may undergo deformation during a surgery (e.g., due to the breathing of the patient), making it even more difficult to obtain accurate 3D information.

Thus, there is a need for a solution that addresses these challenges.

### SUMMARY

The teachings disclosed herein relate to methods, systems, and programming for information management. More particularly, the present teaching relates to methods, systems, and programming related to content summarization.

In one example, a method is disclosed for generating a point cloud of a target organ using a camera and a laser emitter inserted into a patient's body near the target organ and tracked by a tracker. The laser emitter emits, at tracked moving locations, laser beams that hit the target organ at multiple points which are captured by the camera in an image. A three-dimensional (3D) coordinate for each point is determined based on a laser line equation, connecting the laser emitter and the point, and an image line equation, connecting the camera and the point. The 3D coordinates for the multiple points are used to generate the point cloud.

In a different example, a system is disclosed for generating a point cloud of a target organ. The system includes a tracker, a camera, a laser emitter, and a three-dimensional (3D) point cloud determiner. The tracker is deployed in a tracker coordinate system in a surgery for operating on the target organ of a patient. The camera and the laser emitter are inserted into the patient near the target organ so that laser beams embittered by the laser emitter at different tracked moving positions hit the surface of the target organ at respective points which are captured by the camera in an image. With respect to each of the points, the 3D point cloud determiner determines a 3D coordinate in the tracker space based on a laser line equation of a laser line connecting the laser emitter and the respective point and an image line equation of an image line connecting the camera and the respective point. Both the laser line equation and the image line equation are expressed in the tracker space. The point cloud is then generated based on the 3D coordinates of the multiple points representing a depth map.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for generating a point cloud of a target organ using a camera and a laser emitter inserted into a patient's body near the target organ and tracked by a tracker. The laser emitter emits, at tracked moving locations, laser beams that hit the target organ at multiple points which are captured by the camera in an image. A three-dimensional (3D) coordinate for each point is determined based on a laser line equation, connecting the laser emitter and the point, and an image line equation, connecting the camera and the point. The 3D coordinates for the multiple points are used to generate the point cloud.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 shows an exemplary setup in a surgery room to acquire a fitted 3D model of an organ via a tracker, a camera, and a laser emitter working in concert, in accordance with an embodiment of the present teaching;

FIGS. 2A-2D depict exemplary configurations of a camera, a laser emitter, and a tracker with respect to a target object to acquire surface depth information of the target, in accordance with an embodiment of the present teaching;

FIG. 3A shows a diffractive optical element (DOE) used in connection with a laser emitter to obtain diffracted light beams;

FIG. 3B shows a setup incorporating a DOE with a laser emitter to acquire a point cloud of a target object surface, in accordance with an embodiment of the present teaching;

FIG. 3C illustrates an operational mode of obtaining a point cloud of an object based on laser beams emitted by a laser emitter at varying positions, in accordance with different embodiments of the present teaching;

FIGS. 4A-4D visually illustrates a process of obtaining a fitted 3D surface of a target organ, in accordance with an embodiment of the present teaching;

FIG. 5A depicts an exemplary high-level system diagram of a framework for controlling a robot arm to reach a location on a target organ based on a 3D organ model fitted with an acquired real-time point cloud of the target organ, in accordance with an embodiment of the present teaching;

FIG. 5B shows different calibrations to be performed to facilitate an operation of acquiring a point cloud in accordance with an embodiment of the present teaching;

FIG. 6A is a flowchart of an exemplary pre-surgery process to carry out calibration and establishing a 3D model for a target organ, in accordance with an embodiment of the present teaching;

FIG. 6B is a flowchart of an exemplary in-surgery process to obtain a fitted 3D model in alignment with an observed target organ and control of a robot arm to approach the target organ, in accordance with an embodiment of the present teaching;

FIG. 7A depicts an exemplary high-level system diagram of a 3D point cloud determiner, in accordance with an embodiment of the present teaching;

FIG. 7B depicts a scenario in estimating a 3D depth of a point on a target object and an exemplary resolution thereof, in accordance with an embodiment of the present teaching;

FIG. 7C is a flowchart of an exemplary process of a 3D point cloud determiner, in accordance with an embodiment of the present teaching;

FIG. 8 is an illustrative diagram of an exemplary mobile device architecture that may be used to realize a specialized system implementing the present teaching in accordance with various embodiments; and

FIG. 9 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well-known methods, procedures, components, and/or systems have been described at a relatively high-level, without detail, in order to avoid unnecessarily obscuring aspects of the present teachings.

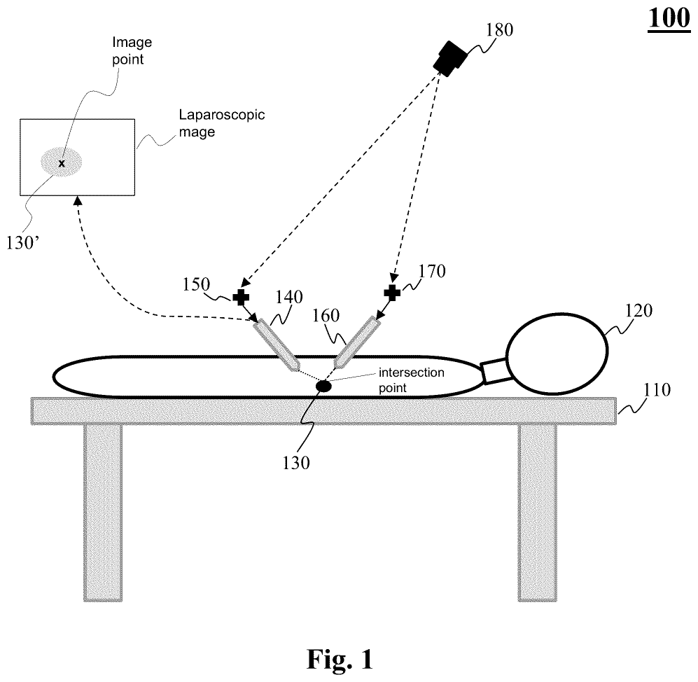

The present teaching discloses exemplary methods, systems, and implementations for acquiring a 3D model for a target organ fitted with real-time acquired depth map of the target organ to enable effective control of a robot arm to approach any 3D location on the surface of the target organ during a surgery. In some embodiments, the fitted 3D model is derived by transforming a 3D model initially obtained for the same target organ to fit a point cloud acquired in real-time from the target organ as observed during a medical procedure. According to the present teaching, a point cloud represents a depth map of, e.g., a part of the surface of a target organ as observed during a medical procedure (e.g., via a laparoscopic camera inserted into a patient's body) and is obtained through, e.g., triangulation between a camera and a laser emitter configured to operate according to the present teaching. FIG. 1 shows an exemplary setup 100 during a medical procedure, in accordance with an embodiment of the present teaching. As seen in FIG. 1, a patient 120 on a surgical table 110 is undergoing a medical procedure to, e.g., remove a tumor located in a target organ (e.g., a liver) or a part thereof 130. The setup includes a tracker system 180, a first device for inserting a camera 140 into a patient's body to acquire a laparoscopic image, and a second device for inserting a laser emitter 160. The camera 140 and the laser emitter 160 are deployed to work in concert with the tracker system 180 to obtain a point cloud of the target organ and accordingly acquire a fitted 3D model that aligns and matches with the organ as observed in the procedure.

According to the present teaching, to obtain a 3D model fitted in alignment with the target organ 130 to enable a robot (or robot arm) to control a surgical instrument to reach a certain location of the target organ, both the camera 140 and the laser emitter 160 are inserted into the patient's body and operate therein. Both the camera 140 and the laser emitter 160 may move around inside the patient's body independently in a restrictive space around. In this configuration, the camera 140 and the laser emitter 160 may have, respectively, a marker attached thereon, i.e., a marker 150 on camera 140 and a marker 170 on emitter 160. The tracker system 180 may also be previously calibrated so that what is observed in the field of view of the tracker system 180 has a known calibrated 3D position in the tracker coordinate system. Given that, the tracker system 180 may track the position of the markers 150 and 170, through which, the positions of the camera 140 and the laser emitter 160 in the tracker coordinate system may be determined given that the rigid spatial relationships between the camera marker 150 and the camera 140 we well as that between the emitter marker 170 and the emitter 160 have been previously calibrated and known. As such, for each tracked location of the camera marker 150, a 3D position of the camera 140 in the tracker space may be accordingly computed. Similarly, using a tracked 3D location of the emitter marker 170, the 3D position of the emitter 160 in the tracker space may be determined. Through mechanism of the tracker system 180, the movement of the camera 140 and/or that of the laser emitter 160 may be tracked in real-time and their dynamic positions can also be determined according to what is discussed herein.

In some embodiments, the camera 140 has its own coordinate system. As the camera 140 and its marker 150 are configured with a fixed spatial relationship, the camera marker 150 has a position in this camera marker space. The camera 140 may be calibrated so that in a 2D image 130′ (e.g., corresponding to the target organ 130) acquired by the camera, each pixel therein has a corresponding 3D coordinate in the camera marker space based on the calibration parameters. A camera viewing line (or image line) may be formed between the 3D coordinate corresponding to the 2D pixel and a point on a target organ (in 3D) being observed. Both the 3D coordinate corresponding to the 2D pixel, and the image line may be expressed accordingly in the camera marker space. The image line extends indefinitely in the camera marker space, and it intercepts the 3D target organ at some surface point of the organ.

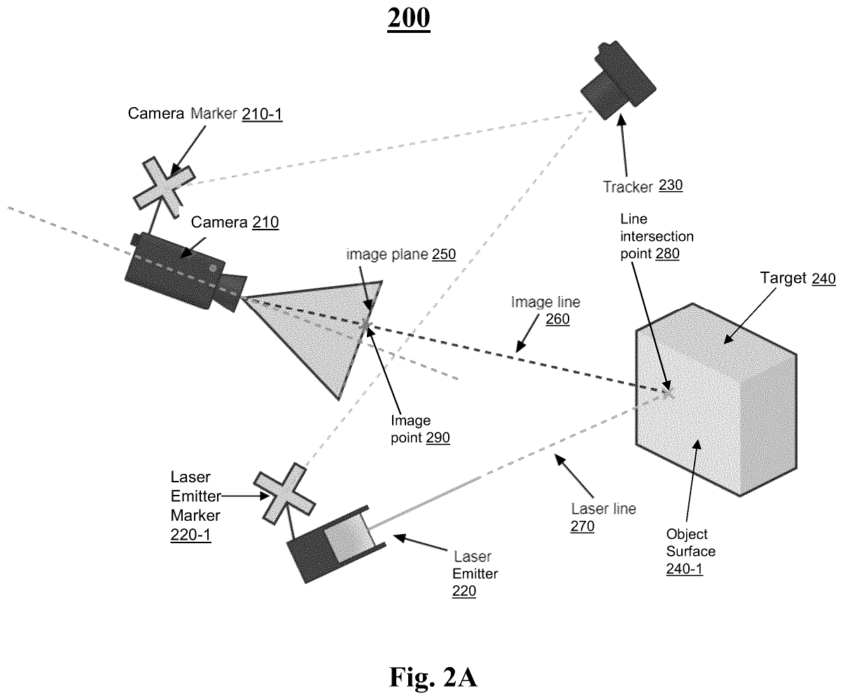

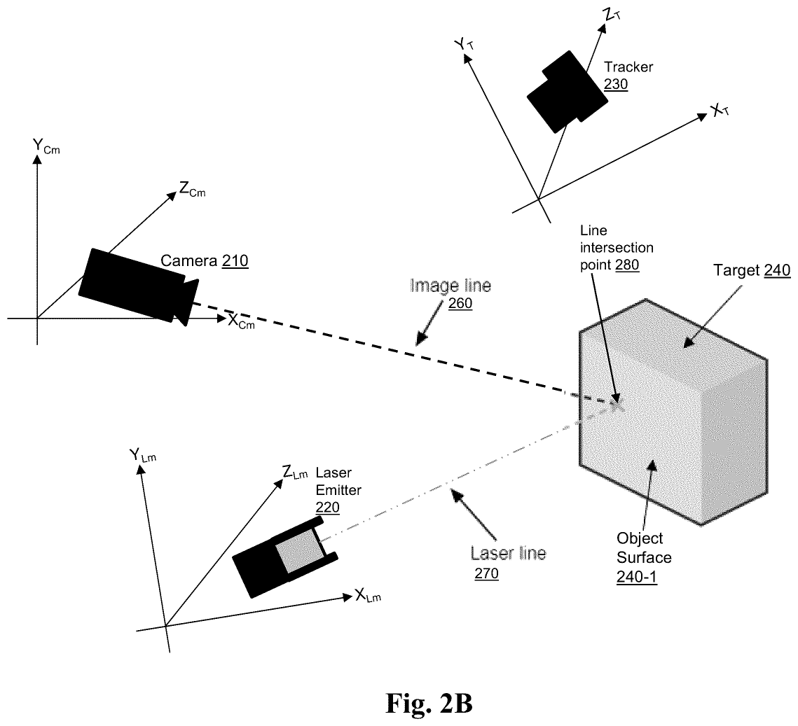

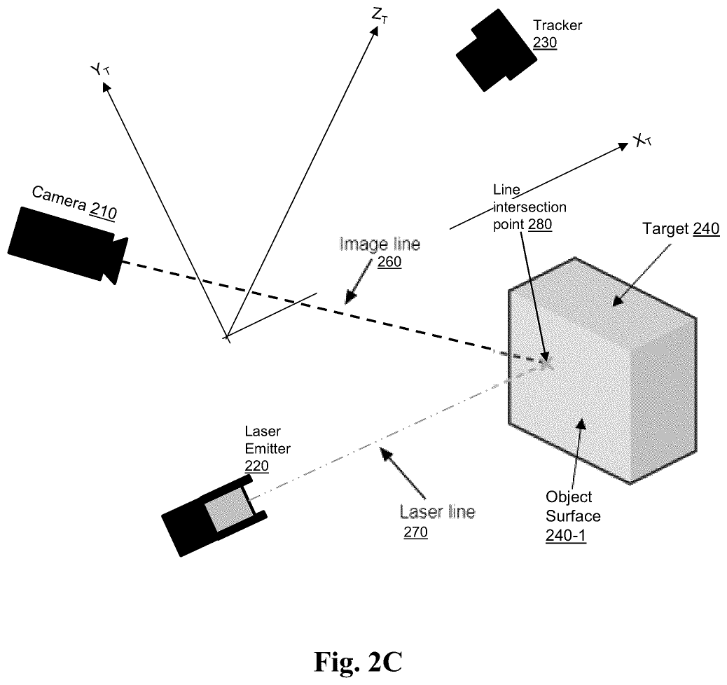

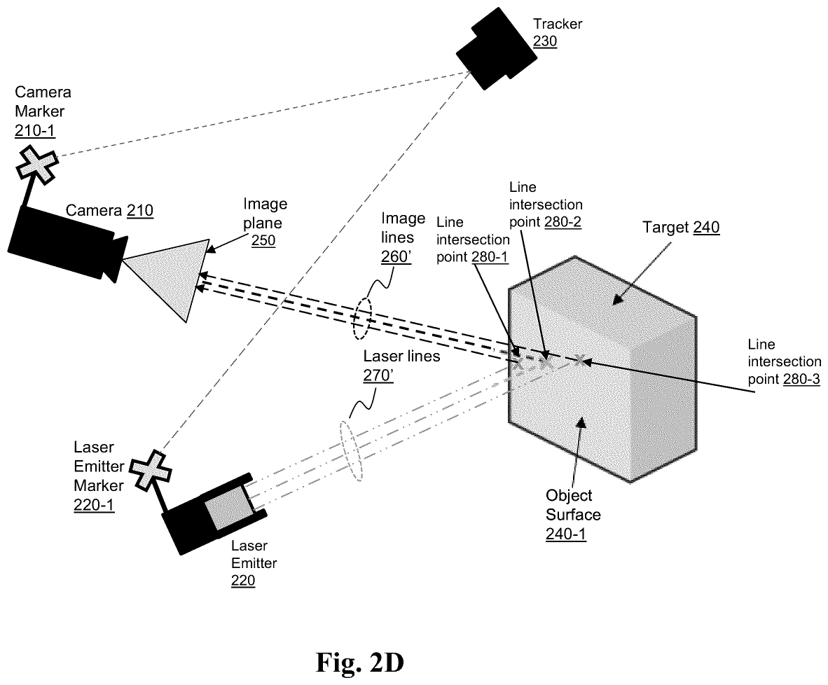

Similarly, the laser emitter 160 may operate in its own coordinate system. Because the laser emitter 160 and the emitter marker 170 thereon have a calibrated spatial relationship, the emitter marker 170 has a position in this laser marker space and so is the laser emitter 160. For each laser beam that the laser emitter 160 emits, a laser line may be formed which may be represented by a laser line equation formulated with respect to the laser marker coordinate system based on parameters associated with the laser emitter 160 as well as calibration parameters. The laser line extends indefinitely in the laser marker space until it hits a 3D object (e.g., a target organ) at some 3D position. The image line and the laser line may intersect on the surface of a target organ, as shown in FIG. 1. The 3D coordinate of the intersection point may be determined according to the present teaching. Details related to detecting intersection points between laser lines and image lines are provided below with reference to FIGS. 2A-4D, 7A-7C.

FIGS. 2A-2D shows exemplary configurations with a camera 210, a laser emitter 220, and a tracker 230 and operations thereof with respect to a target object 240 to acquire surface depth information of the target object 240, in accordance with an embodiment of the present teaching. In FIG. 2A, camera 210 has a camera marker 210-1 affixed thereon and an image plane 250 where the information observed by the camera 210 is formed thereon. The laser emitter 220 has a laser emitter marker 220-1 affixed thereon. Both the camera marker 210-1 and the laser emitter marker 220-1 are tracked by tracker 230. As discussed herein, the camera 210, the laser emitter 220, and the tracker 230 may each be calibrated in their respective coordinate systems as shown in FIG. 2B, i.e., the camera marker space Cm (X_{Cm}, Y_{Cm}, Z_{Cm}), the laser emitter marker space Lm (X_{Lm}, Y_{Lm}, Z_{Lm}), and the tracker space C_{T} (X_{T}, Y_{T}, Z_{T}).

When the laser emitter 220 emits a laser beam onto the object surface 240-1 of the target object 240, the laser beam forms a laser line 270 that hits the object surface 240-1 at a point 280, which may be determined based on the operational parameters of the laser emitter 220. Because the laser emitter 220 is calibrated in its own coordinate system, this laser line 270 may be represented according to an equation expressed with respect to the laser emitter marker space Lm. In general, any line in a 3D space may be characterized by a line equation, which may be determined in different ways. For example, a line may be represented by 2 points Pa, Pb in the 3D space.

$$L ( α \vert {P}_{a} , {P}_{b} ) = α ( \begin{matrix}{X}_{a} \\ {Y}_{a} \\ {Z}_{a}\end{matrix} ) + ( 1 - α ) ( \begin{matrix}{X}_{b} \\ {Y}_{b} \\ {Z}_{b}\end{matrix} ) , α ∈ ℝ$$

The line may also be represented based on a point P (e.g., representing an origin point) and a vector V (e.g., representing a direction):

$$L ( α \vert P , V ) = ( \begin{matrix}{X}_{P} \\ {Y}_{P} \\ {Z}_{P}\end{matrix} ) + α ( \begin{matrix}{X}_{V} \\ {Y}_{V} \\ {Z}_{V}\end{matrix} ) , α ∈ ℝ$$

If we define an X-unit vector as the default direction, then with a 3D pose Ta may be defined as:

$${T}_{a} = [ \begin{matrix}{R}_{{a}_{3 \times 3}} & {P}_{{a}_{3 \times 1}} \\ {0}_{1 \times 3} & 1\end{matrix} ]$$

Define operator ⊗ as

$${T}_{a} ⊗ {P}_{b} = {P}_{{a}_{3 \times 1}} + {R}_{{a}_{3 \times 3}} \times {P}_{b}$$

In some embodiments, the image line and laser line representations in their own coordinate systems can be calibrated by selecting 2 points along the line. For the image line, this may include a camera center point P_{cc|C} and a mirrored image point P_{mi|C}, where “|C” indicates that it is in the camera coordinate system. For the laser line, it can be represented by 2 pre-calibrated points, e.g., P_{t1|L} and P_{t2|L}, where P_{t1|L} may be a point that the beam line reaches a certain distance (e.g., at 100 mm) from the laser lens and P_{t2|L} may be a point where the beam line reaches double of that distance (e.g., at 200 mm) from the laser lens. Similarly, the symbol “|L” indicates that the points are represented in the laser coordinate system. Thus, an equivalent point-vector representation may be obtained for a laser line as follows:

$${L}_{image \vert C} ( α \vert {P}_{cc \vert C} , {V}_{mi \vert C} )$$

$${L}_{laser \vert L} ( β \vert {P}_{t 1 \vert L} , {V}_{t 2 \vert L} )$$

$$where {V}_{mi \vert C} = {P}_{mi \vert C} - {P}_{cc \vert C} , {V}_{t 2 \vert L} = {P}_{t 2 \vert L} - {P}_{t 1 \vert L} .$$

The intersection point 280 may be captured by the camera 210 on the image plane 250 at an image point 290. This is shown in FIG. 2A, with image point 290 on the image plane 250 corresponding to an intercepting point by an image line 260 connecting the focal point of the camera 210 and the point 280 on the target object 240 and the laser line 270. As discussed herein, as the camera is calibrated with respect to its own coordinate system, this image line 260 may be represented according to a line equation expressed with respect to the camera marker space Cm.

As discussed herein, the line intersection point 280 corresponds to a 3D point on the object surface 240-1 of the target object 240. The 3D position of point 280 may be obtained by identifying the intersection point of the image line 260 and the laser line 270. As the equations for these lines are initially formulated in their respective coordinate systems (the camera marker space Cm and the laser emitter marker space Lm), to obtain the 3D coordinate of an intersection point of these lines, their line equations may first be transformed from their respective coordinate systems to a common coordinate system. In general, given a coordinate system A and a coordinate system B, transforming a coordinate in B to A (in other words, get the coordinate in A from a known coordinate in B) can be represented as below:

$${T}_{A}^{B} = [ \begin{matrix}{R}_{{A}_{3 \times 3}}^{B} & {P}_{{A}_{{a}_{3 \times 1}}}^{B} \\ {0}_{1 \times 3} & 1\end{matrix} ]$$

A line representation in coordinate system B can be converted to a line representation in A by:

$${L}_{a \vert A} ( α \vert {P}_{a \vert B} , {V}_{B} ) = {T}_{A}^{B} ⊗ {P}_{a} + α \cdot {R}_{{A}_{3 \times 3}}^{B} \times {V}_{B}$$

To determine the 3D coordinate of the intersecting point 280, the tracker coordinate system C_{T} (X_{T}, Y_{T}, Z_{T}) may be used as the common coordinate system. This may be done via calibration, i.e., calibrating the camera marker space Cm with the tracker space C_{T}, and calibrating the laser emitter marker space Lm with the tracker space C_{T}. This may yield two transformation matrices, one corresponding to a C-T transformation matrix (for transformation between the camera marker space Cm and tracker space C_{T}) and the other an L-T transformation matrix (for transformation between the laser emitter marker space Lm and the tracker space C_{T}).

With tracker calibration, the coordinate conversion between the camera center and the camera's marker (T_{M_C}^{C}) and that between the laser center and the laser's marker (T_{M_L}^{L}) may be calibrated. The poses of the camera marker and the laser marker may be tracked in the tracker space in real-time, denoted as (T_{T}^{M_C}, T_{T}^{M_L}). Therefore, we can get camera and laser's coordinate transformation to tracker space:

$${T}_{T}^{C} = {T}_{M \_ C}^{C} \times {T}_{T}^{M \_ C}$$

$${T}_{T}^{L} = {T}_{M \_ L}^{L} \times {T}_{T}^{M \_ L}$$

To convert the points on both the image line and the laser line to the tracker space, the corresponding line equation for the image line and the line equation for the laser line in the tracker space may be respectively obtained as:

L_{image|T}(α|P_{cc|C},V_{mi|C})

L_{laser|T}(β|P_{t1|L},V_{t2|L})

With the derived C-T transformation matrix, the image line equation for the image line 260 expressed in Cm may be transformed into an image line equation expressed in C_{T}. Similarly, the laser line equation for laser line 270 in Lm may be transformed into a laser line equation in the tracker space C_{T}. This is illustrated in FIG. 2C, where the image line 260 and the laser line 270 are both expressed with respect to the tracker space C_{T}. When both the image line and the laser line are now represented in the same tracker space C_{T}, the line intersection point 280 may be determined by, e.g., solving the 3D position coordinates in both equations. In general, to calculate an intersecting point of two 3D lines (L₁(α|P₁,V₁) and L₂(α|P₂,V₂)), a point from one line that is closest to the other line may be determined:

$${R}_{i} = {\vert {V}_{i} \vert}^{2} , i = 1 , 2$$

$${D}_{4321} = {x}_{{V}_{2}} {x}_{{V}_{1}} + {y}_{{V}_{2}} {y}_{{V}_{1}} + {z}_{{V}_{2}} {z}_{{V}_{1}}$$

$${D}_{3121} = ( {x}_{{p}_{2}} - {x}_{{p}_{1}} ) {x}_{{V}_{1}} + ( {y}_{{p}_{2}} - {y}_{{p}_{1}} ) {y}_{{V}_{1}} + ( {z}_{{p}_{2}} - {z}_{{p}_{1}} ) {z}_{{V}_{1}}$$

$${D}_{4331} = {x}_{{V}_{2}} ( {x}_{{p}_{2}} - {x}_{{p}_{1}} ) + {y}_{{V}_{2}} ( {y}_{{p}_{2}} - {y}_{{p}_{1}} ) + ( {z}_{{p}_{2}} - {z}_{{p}_{1}} ) {z}_{{V}_{1}}$$

$${α}_{1 \_ min} = \frac{{D}_{4321} \times {D}_{4331} - {R}_{2} \times {D}_{3121}}{{D}_{4321}^{2} - {R}_{1} \times {R}_{2}}$$

$${α}_{2 \_ min} = \frac{{R}_{1} \times {D}_{4331} - {D}_{3121} \times {D}_{4321}}{{D}_{4321}^{2} - {R}_{1} \times {R}_{2}}$$

Then the intersection point may be calculated as:

$${P}_{intersect} = \frac{{L}_{image \vert T} ( {α}_{1 \_ min} \vert {P}_{cc \vert C} , {V}_{mi \vert C} ) + {L}_{laser \vert T} ( {α}_{2 \_ min} \vert {P}_{t 1 \vert L} , {V}_{t 2 \vert L} )}{2}$$

Such an intersection point 280 of two lines represented in the tracker space is represented by a 3D coordinate in the tracker space C_{T}. With the mechanism disclosed herein, when the laser emitter 220 operates to emit multiple laser beams, as shown in FIG. 2D, a group of laser lines 270′ may be formed that intercept the object surface at multiple points, i.e., 280-1, 280-2, . . . , 280-3. The multiple laser lines in 270′ may be characterized via equations expressed initially in the laser space Lm and then transformed to the tracker space C_{T} using the L-T transformation matrix. On the other hand, there are multiple image pixels formed on the image plane 250 corresponding to the points hit by the multiple laser beams on the surface of the target object 240. As such, a group of image lines 260′ may be derived and transformed via the C-T transformation matrix to the tracker space C_{T}. The transformed image line equations for 260′ and the transformed laser line equations for 270′ in the common tracker space C_{T} may then be used to determine multiple 3D intersection points (e.g., 280-1, 280-2, . . . , 280-3) on the object surface 240-1.

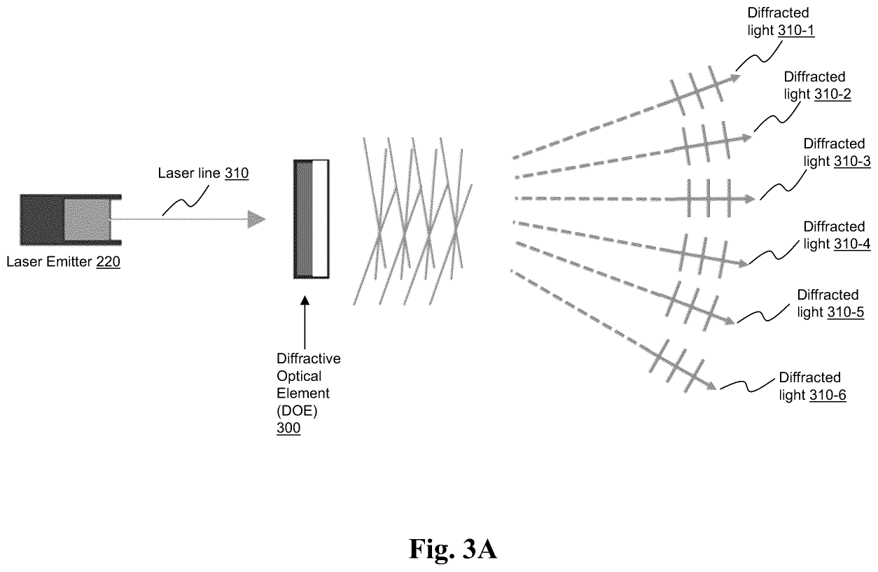

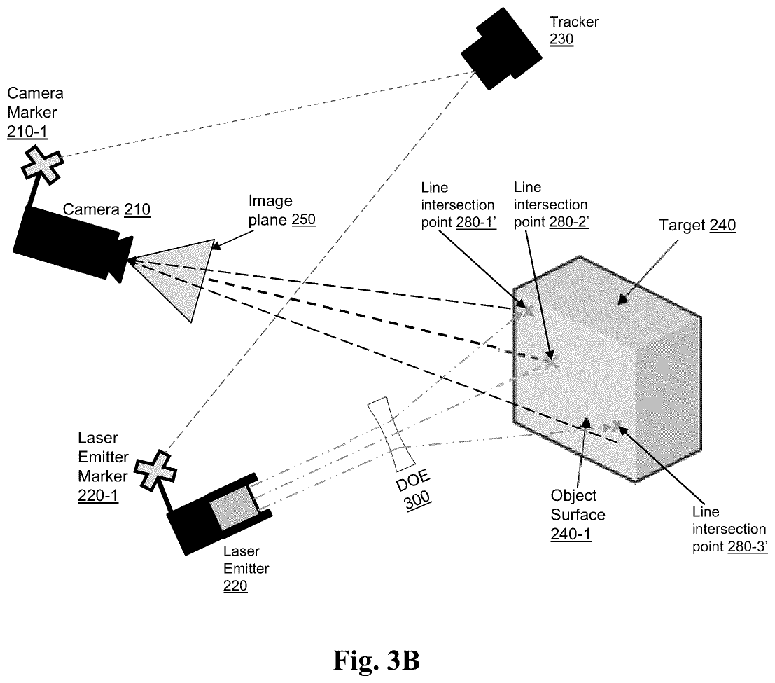

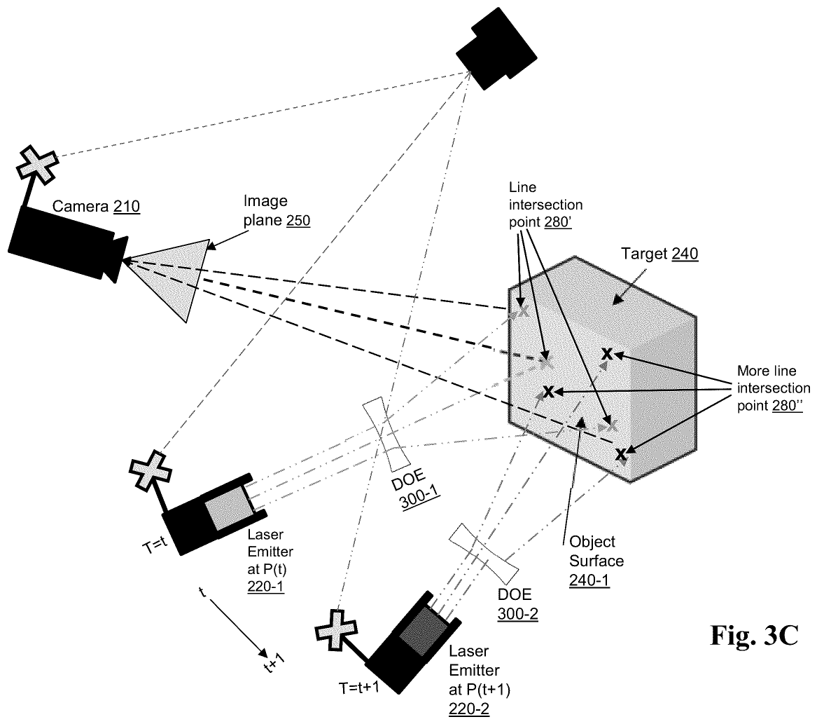

With this mechanism, when there is a sufficient number of laser beams with an adequate dense coverage on the surface of the target object 240, a point cloud for the target object surface may be obtained. FIGS. 3A-3C illustrates some exemplary schemes according to the present teaching to provide the means to obtain a point cloud with a needed scope or coverage as well as sufficiently dense point distribution to adequately capture the characteristics of the depth information of the surface of the target object 240. In some embodiments, to improve the spread of the laser beams, a diffractive optical element (DOE) may be employed together with the laser emitter 220. FIG. 3A illustrates that when a DOE 300 is used in connection with a laser emitter, it may spread a single laser beam into multiple laser light beams. As shown, a DOE 300 may be placed along the path of laser beams emitted by the laser emitter 220, when each laser beam, e.g., 310 as emitted by the laser emitter 220 goes through the DOE 300, is diffracted into multiple laser light beams, e.g., 310-1, . . . , 310-6. As each single laser beam from the laser emitter may be split into multiple laser beams using DOE 300. This may yield not only a wider coverage on an intercepting surface but also with a much dense distribution.

FIG. 3B shows an implementation that incorporates a DOE 300 with the laser emitter 220 in a configuration to work in junction with camera 210 and tracker 230 to acquire a dense point cloud with depth information of a target object, in accordance with an embodiment of the present teaching. Each of the laser beams emitted by the laser emitter 220 goes through the DOE 300 to yield multiple diffracted laser beams. As seen, the laser light spread, e.g., 2810-1′, 280-2′, . . . , 280-3′ in FIG. 3B represents a wider coverage on the object surface than that (280-1, 280-2, . . . , 280-3) as shown in FIG. 2D. As such, the camera 210 is able to capture the image of a larger surface area of the target object 240. Consequently, more depth information may be obtained for a larger surface area on the target object in accordance with the embodiment of the present teaching. In addition, the use of DOE 300 also enables a denser point cloud as more laser beams are created by diffraction which leads to more intersection points. When the operational parameters of the DOE 300 are known, each diffracted laser line from the DOE 300 may be characterized by a laser line equation formulated in accordance with the operational parameters associated with the DOE 300. Each laser line equation for a diffracted laser beam may be expressed in the laser space Lm but can be transformed to that in the common space C_{T}, as discussed herein.

A point cloud with more densely distributed points can characterize more accurately a surface. While the use of the DOE 300 as illustrated above may improve the scope and increase the number of points in a point cloud, additional means may be adopted to further increase both the scope and density of a point cloud. FIG. 3C illustrates an exemplary embodiment of acquiring a point cloud based on laser beams emitted by a laser emitter at varying positions at different times, in accordance with different embodiments of the present teaching. In this illustrated embodiment, the laser emitter 220 coupled with the DOE 300 may be moved around at different times, e.g., at time t, t+1, . . . , etc. At time instant t, the laser emitter 220-1 emits laser beams onto the DOE 300-1, which are diffracted to form more laser lights with a wider spread to hit the object surface 240-1 with a collection of line intersection points 280′. When the laser emitter 220 is moved at t+1 to a different location, the laser emitter 220-2 emits laser beams onto the DOE 300-2, which are diffracted to form more laser lights with a wider spread to hit the object surface 240-1 with a collection of line intersection points 280″. Through this mechanism, not only the number of line intersection points is increased as the intersection points in 280′ and that in 280″ may all be used in the point cloud, but also the scope of the point cloud is also improved as the laser beams emitted at each different location may reach an area on a surface that is not covered by laser beams emitted at other locations.

As discussed herein, in some applications such as a medical procedure, the present teaching may be applied to fit a previously constructed 3D model for a target organ (e.g., a 3D model for the liver of a patient based on previously acquired medical data such as C_{T} scans) to a point cloud generated from the target organ during a surgery with the mechanism as disclosed herein (i.e., via a laparoscopic camera 210 and a laser emitter 220 with a DOE 300 inserted into a patient's body). The point cloud so acquired may include generally the surface of the target organ facing the laparoscopic camera and the laser emitter. In the meantime, the 3D model for the target organ models the entire volume of the target organ (as well as all the internal anatomical structures in the target organ). Given that, fitting the 3D model to the point cloud generated in real-time during the surgery may provide important information for offering useful guidance to the operation, including the orientation of the target organ, the deformation, as well as the part of the target organ that is not visible or reachable by the laparoscopic camera and the laser lights. By fitting the 3D models to the point cloud, the depth information of the entire surface of the target organ can be estimated. With this mechanism according to the present teaching, although a target organ may deform during a surgery, the fitted 3D model can provide a good approximation of the entire target organ, especially the part that is visible by the laparoscopic camera and the laser emitter.

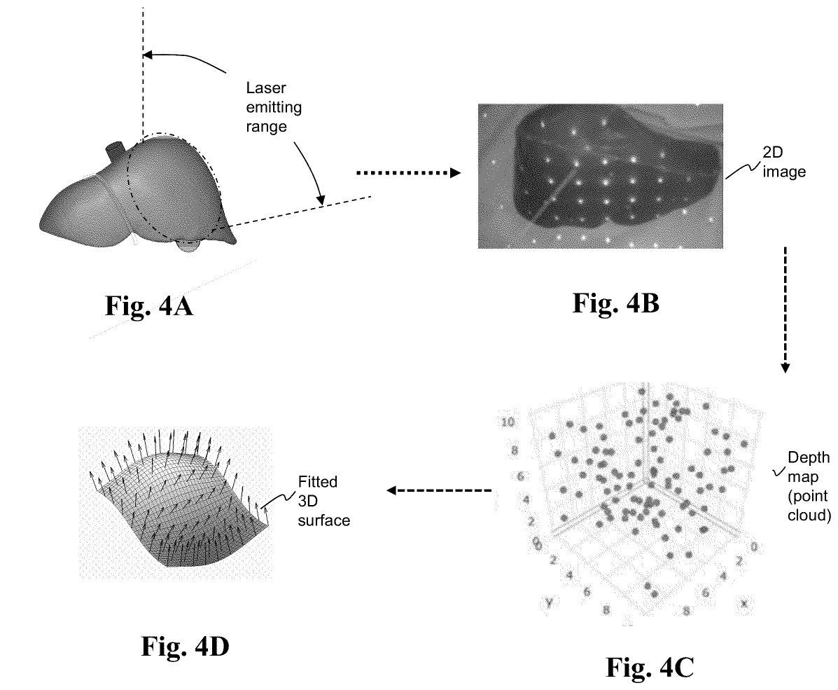

FIGS. 4A-4D depicts visually a process of obtaining a fitted 3D surface of a target organ, in accordance with an embodiment of the present teaching. FIG. 4A shows a 3D model for a target organ, e.g., a liver, where only a part thereof is exposed to the laparoscopic camera and the laser emitter (e.g., the surface of the target organ visible by the camera and within a laser emitting range). That is, the only depth information that may be acquired via the present teaching is for the part of the target organ within the operation range of the present teaching. By applying the point cloud acquisition mechanism according to the present teaching, FIG. 4B shows a 2D laparoscopic image acquired by a laparoscopic camera, which not only shows the target organ in view but also the pixel points (dots) corresponding to observed intersection points corresponding to the hits on the target organ by the laser beams. As discussed herein, for each of the pixels corresponding to the projections of the intersection points, an image line is determined, with a line equation derived with respect to space Cm and then is transformed into an image line equation with respect to the tracker space C_{T}. Similarly, for each laser beam, a laser line equation is obtained with respect to space Lm and then is transformed to a laser line equation with respect to the tracker space C_{T}. For the laser emission pixel points observable in the 2D image as shown in FIG. 4B, the 3D coordinates of the line intersection points are obtained according to the present teaching to form a point cloud or depth map as shown in FIG. 4C.

The point cloud as illustrated in FIG. 4C is then used to fit with the 3D model for the target organ as shown in FIG. 4A to determine a fitted 3D surface as illustrated in FIG. 4D. In some embodiments, the fitting process is to determine a transformation of the 3D model so that a part of the transformed 3D model fits the point cloud. The fitted 3D surface corresponds to a part of the surface of the target organ as modeled by the 3D model and within the laser emitting range. In some embodiments, the fitting process yields a transformation (position translation and orientation) that orients the 3D model in a way so that the portion of the 3D model within the laser emitting range best matches with the point cloud. In this way, the oriented 3D model via this fitting may be used to predict the depth information anywhere on the target organ, which may then be used as a guide to control a robot arm to manipulate a surgical instrument to approach any part of the target organ during the surgery.

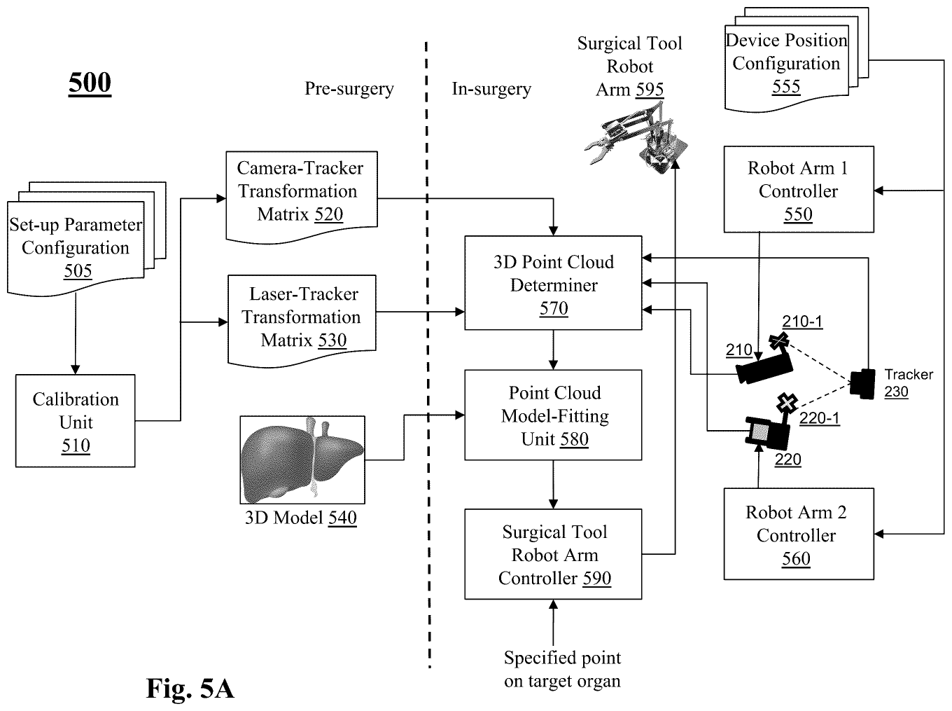

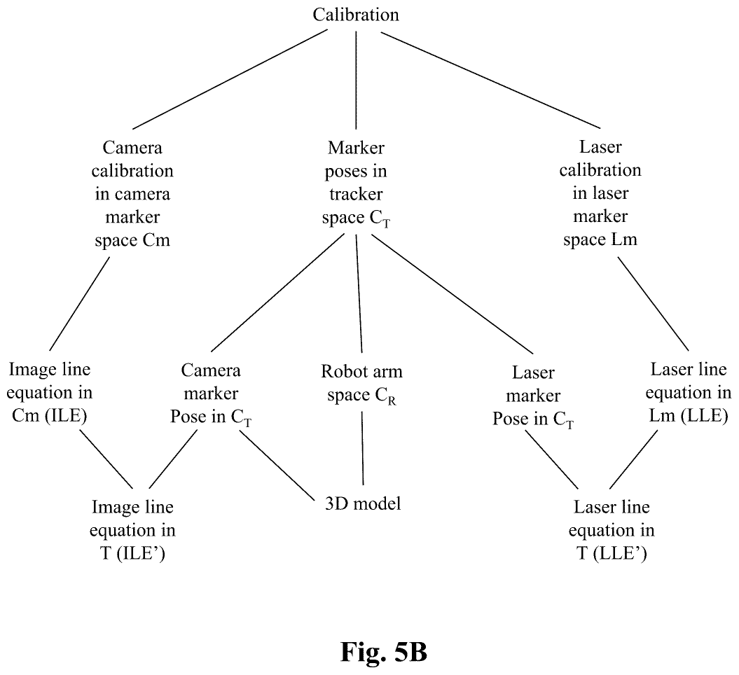

FIG. 5A depicts an exemplary high-level system diagram of a framework 500 for facilitating a robot arm to reach a target organ based on a 3D organ model fitted with a point cloud of the target organ acquired in real time, in accordance with an embodiment of the present teaching. This illustrated framework 500 includes two portions, corresponding to pre-surgery and in-surgery operations. In the pre-surgery part, different calibrations are performed to obtain the transformation matrices as discussed herein that are needed to facilitate the real time operation to estimate a point cloud based on camera and laser data. As shown in FIG. 5A, during a surgery, a laparoscopic camera 210 and a laser emitter 220 with a respective tracker 210-1 and 220-1 coupled thereon, and a tracker 230 are deployed in the surgery room. The camera 210 and the laser emitter 220 may be inserted in a patient's body having their respective trackers remaining outside of the patient's body so that they can be tracked by the tracker 230. Using such a setting, a point cloud associated with a target organ of the patient, e.g., a liver, may be obtained according to the present teaching.

As discussed herein, before a surgery, various calibrations are needed. FIG. 5B illustrates different calibrations that may be performed to facilitate an operation of acquiring a point cloud in accordance with an embodiment of the present teaching. As shown in FIG. 5B, this includes calibration of the camera 210 in camera marker space Cm (so that each pixel in a 2D image acquired by the camera 210 corresponds to a 3D coordinate and an image line expressed in Cm), the calibration of laser emitter 220 in laser emitter marker space Lm (so that each emitted laser beam has a corresponding laser line equation expressed in Lm). The tracker 230 is in a tracker space C_{T} and tracker 230 has a calibrated position in C_{T}. In addition, the needed calibration also includes to obtain the C-T transformation matrix 520 (for transformation between the camera marker space Cm with respect to the tracker space C_{T}) as well as the L-T transformation matrix 530 (for transformation between the laser emitter marker space Lm with respect to the tracker space C_{T}). Using such transformation matrices, an image line equation ILE in Cm may be transformed in the tracker space C_{T} to derive an ILE′. Similarly, a laser line equation or LLE in Lm may be transformed to obtain an LLE′ in C_{T}.

The tracker space C_{T} may also be calibrated with respect to the coordinate systems of the robot arms so that the 3D information in C_{T} associated with a point cloud may be transformed into the 3D information in the robot arm space, say C_{R}, to facilitate the control of the robot arms to reach any point on the target organ according to the 3D information expressed in C_{R}. When the previously constructed 3D model is fit against the point cloud in C_{T} to obtain a fitted 3D model in C_{T}, to enable the control of a robot arm to manipulate a surgical instrument to approach anywhere of a target organ (now modeled by the fitted 3D model), the fitted 3D model may also be transformed from C_{T} to C_{R}.

To carry out these calibrations, the pre-surgery portion of framework 500 as shown in FIG. 5A includes a calibration unit 510 provided to perform various calibrations prior to a surgery to derive, using the set-up parameter configuration 505, corresponding transformation matrices, including a camera-tracker (C-T) transformation metric 520 and a laser-tracker (L-T) transformation matrix 530 (for transforming an ILE in Cm to a corresponding ILE′ in C_{T} as well as an LLE in space Lm to an LLE′ in C_{T}, as discussed herein). In addition, a 3D model 540 is obtained at the pre-surgery stage for a target organ based on, e.g., medical data (e.g., C_{T} scan) of the patient in, e.g., its own model coordinate space. In some embodiments, during the fitting process in a surgery, the 3D model 540 may first be transformed from its initial model space to C_{T} and then the dynamic 3D model fitted to a point cloud of the target organ may be transformed again into the robot arm space to enable the control of the robot arm.

At the surgery stage, the C-T and L-T transformation matrices (520 and 530) as well as the 3D model 540 are utilized to obtain a fitted 3D model based on a point cloud generated via triangulation in a setting with a laparoscopic camera 210, a laser emitter 220, and a tracker 230, in accordance with the present teaching. The laser emitter 220 in a surgery room may be deployed with a DOE so that each laser beam emitted can yield a plurality of diffracted laser beams hitting the surface of a target organ. The in-surgery portion of the framework 500, as shown in FIG. 5A, comprises a robot arm 1 controller 550 for controlling the movement of the laparoscopic camera 210, a robot arm 2 controller 560 for controlling the movement of the laser emitter 220, a 3D point cloud determiner 570, a point cloud model fitting unit 580, and a surgical tool robot arm controller 590 for manipulating a surgical tool robot arm 595. During the surgery, the robot arm 1 controller 550 and the robot arm 2 controller 560 may be controlled to insert, respectively, the laparoscopic camera 210 and the laser emitter 220 into a patient's body to reach some specified positions near a target organ, which are determined so that the laser beams emitted by the laser emitter 220 may reach the surface of the target organ and target organ is within the field of view of the laparoscopic camera 210 to capture the intersecting points by the laser beams emitted by the laser emitter 220 (see FIG. 4B).

The 3D point cloud determiner 570 may be provided for creating a point cloud by determining the line intersection points 280 (including 280′, 280″, etc.) as described herein in accordance with the present teaching. The intersection points in the point cloud represented in the tracker space C_{T} may then be used by the point cloud model fitting unit 580 to fit the previously constructed 3D model 540 to the points in the point cloud to yield a fitted 3D model in the tracker space C_{T}, which may further be transformed into the surgical tool robot arm space C_{R} to enable the surgical tool robot arm controller 590 to control the surgical tool robot arm 595 to approach any of the surface point on the target organ. The surgical tool robot arm controller 590 may be provided to take an instruction specifying a 3D position for a surgical instrument to reach and accordingly issues commands to manipulate the surgical tool robot arm to move the surgical instrument towards the specified 3D position. For instance, the specified 3D position may correspond to a surface point on the target organ (e.g., a liver) where a cut (e.g., in a resection operation) is to be made. In this case, the surgical robot arm may be controlled to move a surgical instrument such as a cutter to the surface point to get ready for the incision to be made at that location.

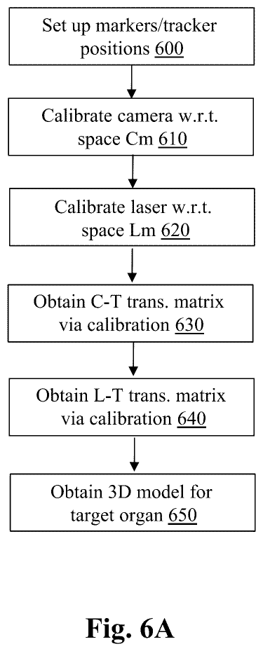

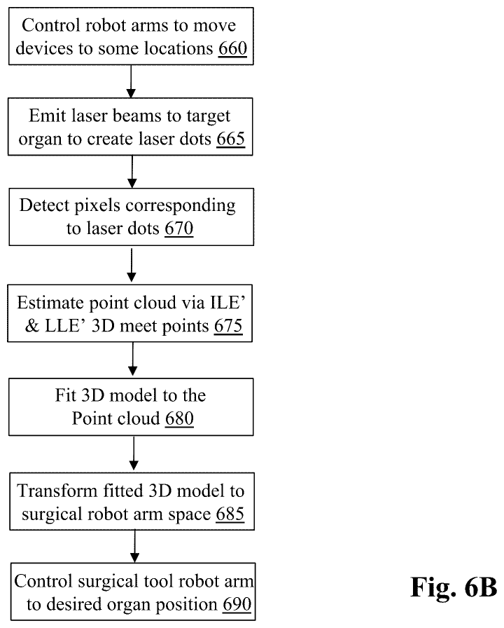

FIG. 6A is a flowchart of an exemplary pre-surgery process to carry out needed calibrations and create the 3D model 540 for a target organ, in accordance with an embodiment of the present teaching. Different devices may be set up, at 600, in the same way as in an actual surgery room for carrying out different calibrations. The set up includes the placement of the camera 210, the laser emitter 220, the tracker 230, as well as attaching markers respectively to the camera 210 and the laser emitter 220. In this setup, the relative spatial relationships between any two devices can be fixed. This includes the spatial relationship between the camera marker 210-1 and the camera 210, the relative spatial relationship between the laser emitter marker 220-1 and the laser emitter 220, the relative placement positions between the tracker 230 and the camera 210 and laser emitter 220.

The calibration unit 510 may then carry out various calibrations, including calibrating, at 610, the camera 210 with respect to the camera marker space Cm and calibrating, at 620, the laser emitter 220 with respect to the laser emitter marker space Lm. In addition, different spaces may also be calibrated so that coordinates in one coordinate system may be transformed into another. For example, the C-T transformation matrix 520 may be obtained via calibration, at 630, and the L-T transformation matrix 530 may be obtained, at 640, by calibrating against the laser marker space and the tracker space. Other calibrations may also be carried out (not shown in FIG. 6A) such as the transformation between the tracker space C_{T} and the robot arm space C_{R} associated with the surgical tool robot arm 595. The 3D model 540 may also be obtained at 650. In some embodiments, the 3D model 540 may be constructed via, e.g., image data acquired from the patient related to the target organ and analysis result thereof to indicate various anatomical structures to be modeled, which may include the shape/volume of the target organ, blood vessels, tumors, tissues, and/or measures characterizing the same. The 3D model 540 for the target organ may be represented in a model coordinate system.

FIG. 6B is a flowchart of an exemplary process for the in-surgery part of framework 500 to control a robot arm to approach a position of a target organ via a fitted 3D model in alignment with the observed target organ, in accordance with an embodiment of the present teaching. During a surgical procedure, the robot arm 1 controller 550 and the robot arm 2 controller 560 may first control, at 660, to insert, respectively, the laparoscopic camera 210 and the laser emitter 220 into a patient's body to arrive at some positions, which may be specified in the device position configuration 555 obtained via, e.g., pre-surgical planning. For instance, such specified positions may be near the target organ as shown in FIG. 2A. The tracker 230 may also be installed at some pre-determined location from where the markers attached on the camera 210 and the laser emitter 220 may both visible within the field of view. Once the setup is complete, the devices may collaborate as discussed herein to acquire a point cloud related to a portion of the surface of the target organ in accordance with the present teaching.

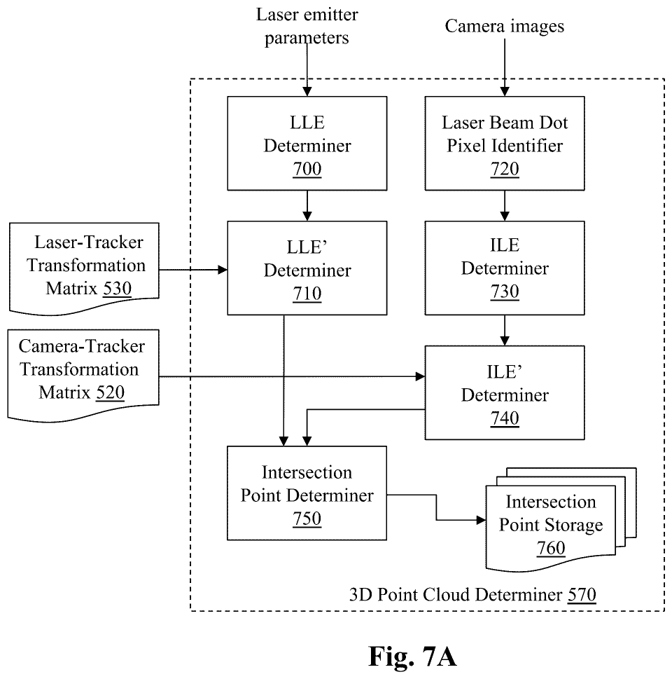

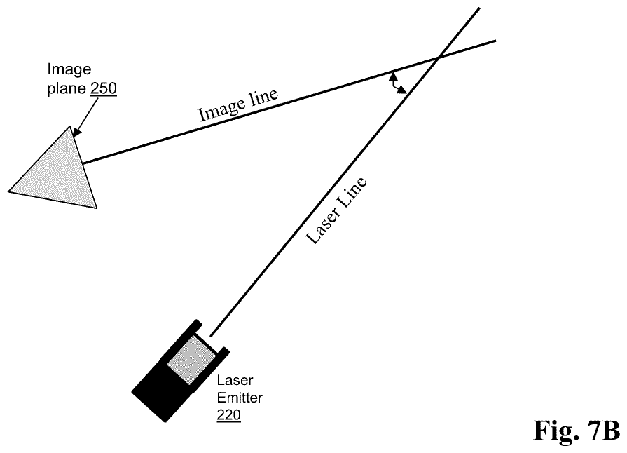

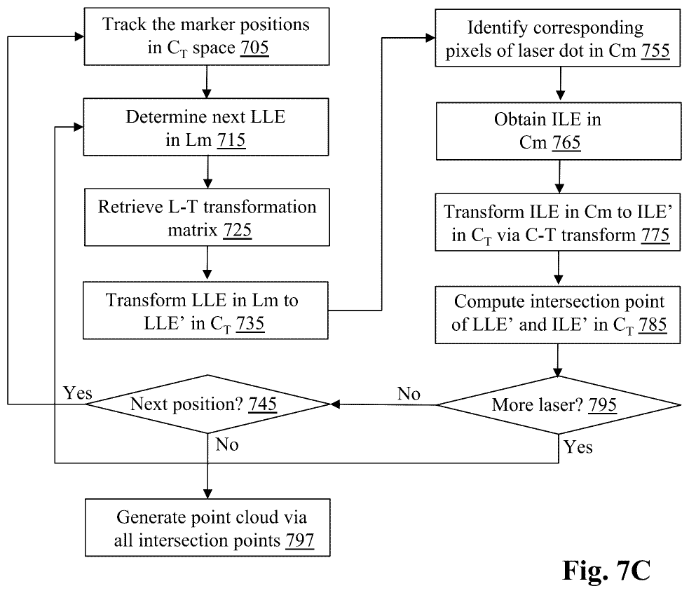

In operation, the laser emitter 220 emits, at 665, laser beams onto the target organ to create laser beam dots on the surface of the target organ. The laparoscopic camera 210 then acquires a 2D image with such laser beam dots therein, which may be detected, at 670, from the captured 2D image at certain pixel locations corresponding to the laser beam dots. Based on the laser beams emitted and the detected pixels corresponding to the laser beam dots in the laparoscopic image, the 3D point cloud determiner 570 estimates, at 675, a point cloud representing an estimated depth map of the part of the target organ surface covered by the laser beams. Details related to the 3D point cloud determiner 570 are provided with reference to FIGS. 7A-7C. The depth information included in this point cloud is represented with respect to the tracker space C_{T}, as discussed herein and is then used, by the point cloud model fitting unit 580, to fit the 3D model 540, at 680, so that the fitted 3D model is aligned with the target organ in C_{T}. To be able to control the surgical tool robot arm 595 to move a surgical instrument to approach any surface point on the target organ, the surgical tool robot arm controller 590 may then transform, at 685, the fitted model from the tracker space C_{T} to the robot arm space C_{R}. When an instruction is received specifying a surface point on the target organ, the surgical tool robot arm controller 590 may compute the kinematic parameters needed to control, at 690, the surgical tool robot arm 595 to move a surgical instrument to the specified surface point on the target organ.

FIG. 7A depicts an exemplary high-level system diagram of the 3D point cloud determiner 570, constructed and operating in accordance with an embodiment of the present teaching. In this illustrated embodiment, the 3D point cloud determiner 570 comprises an LLE (laser line equation) determiner 700, an LLE′ determiner 710, a laser beam dot pixel identifier 720, an ILE (image line equation) determiner 730, an ILE′ determiner 740, and an intersection point determiner 750. The LLE determiner 700 is provided to compute a laser line equation for each laser beam (including each diffracted laser beam when DOE is deployed with the laser emitter). The LLE′ determiner 710 is provided to transform each LLE in Lm space to LLE′, the same laser line equation but expressed in the tracker space C_{T}. The laser beam dot pixel identifier 720 is provided to identify those pixels in the laparoscopic image that correspond to the laser beam dots that the laser beams (or diffracted laser beams) hit on the surface of the target organ. For each of the pixels corresponding to the laser beam dots, the ILE determiner 730 is provided to determine an image line equation or ILE in space Cm and the ILE′ determiner 740 is provided for converting the ILE to ILE′, the image line equation expressed in the tracker space C_{T}. The intersection point determiner 750 is provided for finding an intersection point of each pair of LLE′ and ILE′ in the tracker space C_{T}. Such identified intersection points are stored in intersection point storage 760.

In some situations, an ILE′ and an LLE′ may not intersect exactly. This may happen due to, e.g., the resolution of the image plane. FIG. 7B illustrates an example, where the image line does not exactly meet a laser line. In this case, an approximate of the intersection point may be determined. For instance, a point on the image line and another point on the laser line may be identified which form a shortest distance between the two lines. In this case, a mid-point from a line connecting the two points may be used as an approximated intersection point. In some embodiments, some criterion may be introduced so that only when the shortest distance is below some acceptable range, the approximation may be made. Otherwise, no intersection point is to be obtained through approximation.

As discussed herein, the laser emitter 220 may emit laser beams from different locations (see FIG. 3C). The laser emitter 220 may be moved around via a robot arm control according to, e.g., the device position configuration 555. At each laser emitter position, a plurality of laser beams (including diffracted laser beams through a DOE) may lead to a plurality of LLE's and a corresponding plurality of ILE's so that a group of intersection points may be obtained and stored in intersection point storage 760. As discussed herein, in this manner, via emitting laser beams at multiple locations, more intersection points may be obtained this way. Different groups of intersection points obtained with respect to different laser emitting positions may then be used to form a point cloud with enhanced scope and density.

FIG. 7C is a flowchart of an exemplary process of the 3D point cloud determiner 570, in accordance with an embodiment of the present teaching. During a surgery, the 3D marker positions of the camera marker and the laser emitter marker in C_{T} are tracked at 705 and they may be used to determine the 3D positions of the laparoscopic camera 210 and the laser emitter 220 in C_{T}. When the laser emitter 220 with a DOE emits laser beams, the LLE determiner 700 determines, at 715, an LLE in Lm for each of the laser lines. To convert the LLE in Lm to LLE′ in C_{T}, the L-T transformation matrix 530 is retrieved at 725 and used, at 735, by the LLE′ determiner 710 to transform, at 735, the LLE in Lm to LLE′ in C_{T}. On the other hand, the laparoscopic camera 210 captures a camera image with laser beam dots on the organ surface visible therein. The camera image is processed by the laser beam dot pixel identifier 720 to identify, at 755, each pixel corresponding to a laser beam dot on the surface of the target organ. Based on each of such identified pixels, the ILE determiner 730 obtains, at 765, an ILE in Cm, which is then transformed by the ILE′ determiner 740 to obtain, at 775, an ILE′ in the tracker space C_{T}. Based on the LLE′ and ILE′ in C_{T}, the intersection point determiner 750 computes, at 785, the intersection point of ILE′ and LLE′ and stores it in intersection point storage 760. The process continues until, determined at 795, all intersection points with respect to the laser beams emitted by the laser emitter 220 with the DOE at a current position are computed. When the laser emitter 220 is moved to a different position, determined at 745, the process returns to step 705 until intersection points corresponding to the next groups of laser beams are computed.

As discussed herein, all the intersecting points obtained based on laser beams embittered at different positions, a point cloud can be generated via all intersection points 797 and used, by the point cloud model fitting unit 580, to fit the 3D model 540 to the point cloud to align the 3D model 540 to the actual surface of the target organ in the surgery to derive a fitted 3D model in the tracker space C_{T}, which may then be converted to the surgical tool robot arm space C_{R} to allow the surgical tool robot arm controller 590 to control the surgical robot arm to move a surgical tool to a location on the target organ in the same coordinate system.

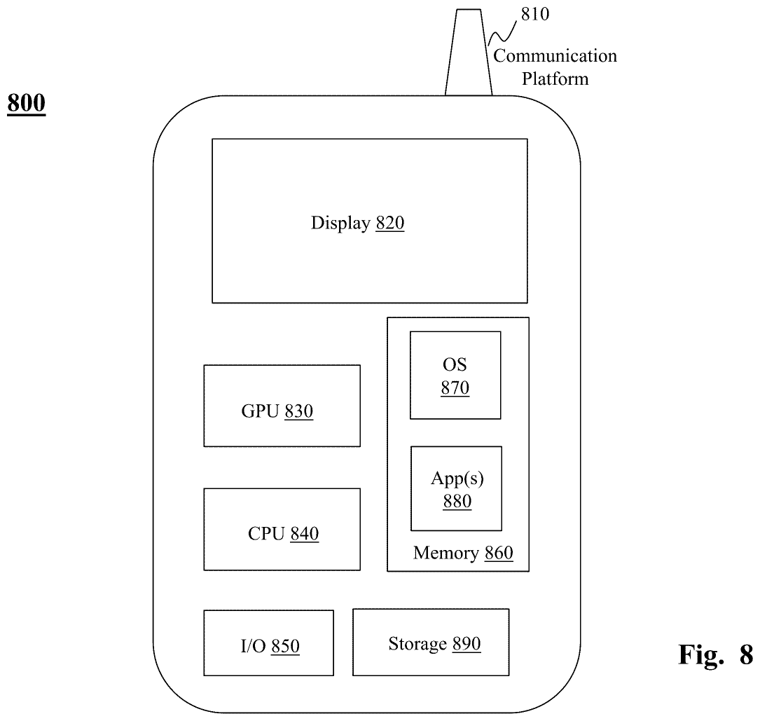

FIG. 8 is an illustrative diagram of an exemplary mobile device architecture that may be used to realize a specialized system implementing the present teaching in accordance with various embodiments. In this example, the user device on which the present teaching may be implemented corresponds to a mobile device 800, including, but not limited to, a smart phone, a tablet, a music player, a handled gaming console, a global positioning system (GPS) receiver, and a wearable computing device, or in any other form factor. Mobile device 800 may include one or more central processing units (“CPUs”) 840, one or more graphic processing units (“GPUs”) 830, a display 820, a memory 860, a communication platform 810, such as a wireless communication module, storage 890, and one or more input/output (I/O) devices 850. Any other suitable component, including but not limited to a system bus or a controller (not shown), may also be included in the mobile device 800. As shown in FIG. 8, a mobile operating system 870 (e.g., iOS, Android, Windows Phone, etc.), and one or more applications 880 may be loaded into memory 860 from storage 890 in order to be executed by the CPU 840. The applications 880 may include a user interface or any other suitable mobile apps for information analytics and management according to the present teaching on, at least partially, the mobile device 800. User interactions, if any, may be achieved via the I/O devices 850 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result, the drawings should be self-explanatory.

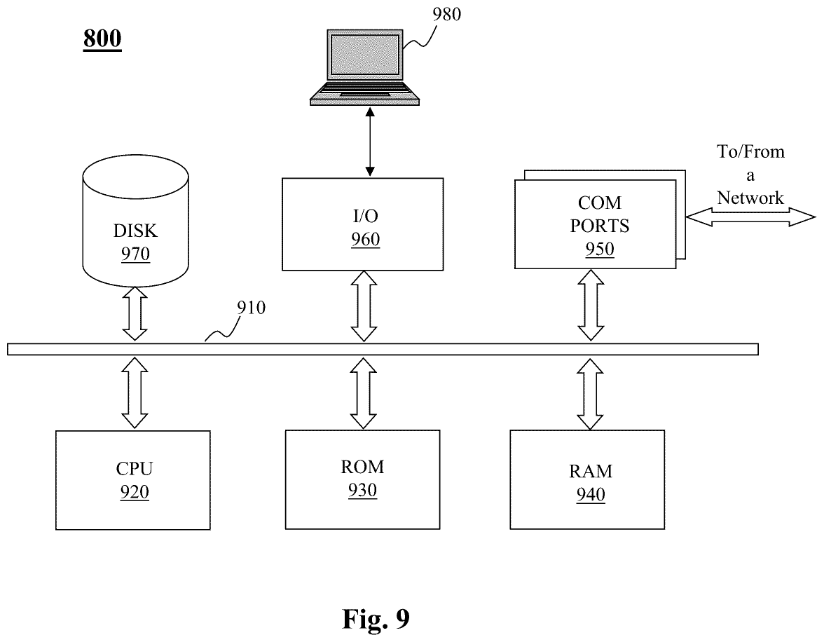

FIG. 9 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special-purpose computer. Both can be used to implement a specialized system for the present teaching. This computer 900 may be used to implement any component or aspect of the framework as disclosed herein. For example, the information analytical and management method and system as disclosed herein may be implemented on a computer such as computer 900, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 900, for example, includes COM ports 950 connected to and from a network connected thereto to facilitate data communications. Computer 900 also includes a central processing unit (CPU) 920, in the form of one or more processors, for executing program instructions. The exemplary computer platform includes an internal communication bus 910, program storage and data storage of different forms (e.g., disk 970, read only memory (ROM) 930, or random-access memory (RAM) 940), for various data files to be processed and/or communicated by computer 900, as well as possibly program instructions to be executed by CPU 920. Computer 900 also includes an I/O component 960, supporting input/output flows between the computer and other components therein such as user interface elements 980. Computer 900 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable the loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or a physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as the main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, a hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
deploying a tracker in a tracker coordinate system in a surgery for operating on a target organ of a patient;
inserting a camera and a laser emitter into the patient near the target organ so that laser beams embittered by the laser emitter at different tracked moving positions hit the surface of the target organ at respective points which are captured by the camera in an image;
with respect to each of the respective points,
deriving a laser line equation of a laser line connecting the laser emitter and the respective point,
deriving an image line equation of an image line connecting the camera and the respective point, wherein the laser line equation and the image line equation are expressed in the tracker coordinate system, and
obtaining a three-dimensional (3D) coordinate of the respective point in the tracker coordinate system based on the laser line equation and the image line equation; and
generating a point cloud based on the 3D coordinates in the tracker coordinate system for the respective points representing a depth map of the captured part of the surface of the target organ.

2. The method of claim 1, wherein
the camera is coupled with a camera marker with a first spatial relation;
the laser emitter is coupled with a laser emitter marker with a second spatial relation, wherein
the camera marker and the laser emitter marker are outside of the patient's body and are tracked by the tracker in the tracker coordinate system,
a tracked 3D location of the camera can be determined from tracked position of the camera marker based on the first spatial relation, and
a tracked 3D location of the laser emitter can be determined from tracked position of the laser emitter marker based on the second spatial relation.

3. The method of claim 2, wherein the step of deriving a laser line equation of a laser line comprises:
determining an initial laser line equation in a laser emitter coordinate system for the laser line based on the tracked 3D location of the laser emitter and operating parameters of the laser emitter; and
converting the initial laser line equation in the laser emitter coordinate system to the laser line equation in the tracker coordinate system based on a laser-tracker transformation matrix obtained via calibration.

4. The method of claim 2, wherein the step of deriving an image line equation of an image line comprises:
determining a two-dimensional (2D) coordinate of a pixel in the image corresponding to a projection of the respective point;
obtaining an initial image line equation based on the 2D coordinate, the tracked 3D location of the camera, and operating parameters of the camera in a camera coordinate system; and
converting the initial image line equation in the camera coordinate system to the image line equation in the tracker coordination system based on a camera-tracker transformation matrix obtained via calibration.

5. The method of claim 1, wherein the 3D coordinate of the respective point in the tracker coordinate system is obtained by:
identifying a meeting coordinate in the tracker coordinate system that satisfies both the laser line equation and the image line equation; and
providing the meeting coordinate in the tracker coordinate system as the 3D coordinate of the respective point.

6. The method of claim 1, wherein the 3D coordinate of the respective point in the tracker coordinate system is obtained by:
identifying a first point on the laser line satisfying the laser line equation and a second point on the image line satisfying the image line equation so that a distance between the first and the second points represents a minimized distance between the laser line and the image line;
selecting an approximate point between the first and the second point along a line connecting the first and the second point; and
obtaining a 3D coordinate of the approximate point in the tracker coordinate system as the 3D coordinate of the respective point.

7. The method of claim 1, wherein the respective points include:
additional hits on the surface by beams from a diffractive optical element (DOE) placed in front of the laser emitter, wherein the DOE yields multiple beams for each of the laser beam from the laser emitter that passes through the DOE;
additional laser beams emitted by the laser emitter at the different tracked moving positions; and
the combination thereof.

8. The method of claim 1, further comprising:
retrieving a 3D model for the target model previously constructed based on data related to the patient; and
fitting the 3D model to the point cloud to obtain a fitted 3D model in the tracker coordinate system that aligns with the target organ and fits the surface of the target organ represented by the point cloud.

9. The method of claim 8, further comprising transforming the fitted 3D model in the tracker coordinate system to a transformed fitted 3D model in a robot coordinate system.

10. The method of claim 9, further comprising:
receiving a specified location on the surface of the target organ during the surgery;
identifying, from the transformed fitted 3D model, a 3D coordinate in the robot coordinate system for the specified location; and
controlling a robot arm holding a surgical instrument to reach the specified location on the surface of the target organ based on the 3D coordinate in the robot coordinated system.

11. A system, comprising:
a tracker deployed in a tracker coordinate system in a surgery for operating on a target organ of a patient;
a camera and a laser emitter inserted into the patient near the target organ so that laser beams embittered by the laser emitter at different tracked moving positions hit the surface of the target organ at respective points which are captured by the camera in an image;
a three-dimensional (3D) point cloud determiner implemented by a processor and configured for, with respect to each of the respective points,
deriving a laser line equation of a laser line connecting the laser emitter and the respective point,
deriving an image line equation of an image line connecting the camera and the respective point, wherein the laser line equation and the image line equation are expressed in the tracker coordinate system, and
obtaining a 3D coordinate of the respective point in the tracker coordinate system based on the laser line equation and the image line equation, and
generating a point cloud based on the 3D coordinates in the tracker coordinate system for the respective points representing a depth map of the captured part of the surface of the target organ.

12. The system of claim 11, wherein
the camera is coupled with a camera marker with a first spatial relation;
the laser emitter is coupled with a laser emitter marker with a second spatial relation, wherein
the camera marker and the laser emitter marker are outside of the patient's body and are tracked by the tracker in the tracker coordinate system,
a tracked 3D location of the camera can be determined from tracked position of the camera marker based on the first spatial relation, and
a tracked 3D location of the laser emitter can be determined from tracked position of the laser emitter marker based on the second spatial relation.

13. The system of claim 12, wherein the step of deriving a laser line equation of a laser line comprises:
determining an initial laser line equation in a laser emitter coordinate system for the laser line based on the tracked 3D location of the laser emitter and operating parameters of the laser emitter; and
converting the initial laser line equation in the laser emitter coordinate system to the laser line equation in the tracker coordinate system based on a laser-tracker transformation matrix obtained via calibration.

14. The system of claim 12, wherein the step of deriving an image line equation of an image line comprises:
determining a two-dimensional (2D) coordinate of a pixel in the image corresponding to a projection of the respective point;
obtaining an initial image line equation based on the 2D coordinate, the tracked 3D location of the camera, and operating parameters of the camera in a camera coordinate system; and
converting the initial image line equation in the camera coordinate system to the image line equation in the tracker coordination system based on a camera-tracker transformation matrix obtained via calibration.

15. The system of claim 11, wherein the 3D coordinate of the respective point in the tracker coordinate system is obtained by:
identifying a meeting coordinate in the tracker coordinate system that satisfies both the laser line equation and the image line equation; and
providing the meeting coordinate in the tracker coordinate system as the 3D coordinate of the respective point.

16. The system of claim 11, wherein the 3D coordinate of the respective point in the tracker coordinate system is obtained by:
identifying a first point on the laser line satisfying the laser line equation and a second point on the image line satisfying the image line equation so that a distance between the first and the second points represents a minimized distance between the laser line and the image line;
selecting an approximate point between the first and the second point along a line connecting the first and the second point; and
obtaining a 3D coordinate of the approximate point in the tracker coordinate system as the 3D coordinate of the respective point.

17. The system of claim 11, wherein the respective points include:
additional hits on the surface by beams from a diffractive optical element (DOE) placed in front of the laser emitter, wherein the DOE yields multiple beams for each of the laser beam from the laser emitter that passes through the DOE;
additional laser beams emitted by the laser emitter at the different tracked moving positions; and
the combination thereof.

18. The system of claim 11, further comprising a point cloud model-fitting unit implemented by a processor and configured for:
retrieving a 3D model for the target model previously constructed based on data related to the patient; and
fitting the 3D model to the point cloud to obtain a fitted 3D model in the tracker coordinate system that aligns with the target organ and fits the surface of the target organ represented by the point cloud.

19. The system of claim 18, wherein the point cloud model-fitting unit is further configured for transforming the fitted 3D model in the tracker coordinate system to a transformed fitted 3D model in a robot coordinate system.

20. The system of claim 19, further comprising a surgical tool robot arm controller implemented by a processor and configured for:
receiving a specified location on the surface of the target organ during the surgery;
identifying, from the transformed fitted 3D model, a 3D coordinate in the robot coordinate system for the specified location; and
controlling a robot arm holding a surgical instrument to reach the specified location on the surface of the target organ based on the 3D coordinate in the robot coordinated system.
